(19) United States
(12) Patent Application Publication
(10) Pub. No.: US 2024/0008713 A1
(43) Pub. Date: Jan. 11, 2024

(54) IMAGE PICKUP UNIT AND ENDOSCOPE

(71) Applicant: OLYMPUS CORPORATION, Tokyo (JP)

(72) Inventor: Hidenori HASHIMOTO, Sagamihara-shi (JP)

(73) Assignee: OLYMPUS CORPORATION, Tokyo (JP)

(21) Appl. No.: 18/371,154

(22) Filed: Sep. 21, 2023

Related U.S. Application Data
(63) Continuation of application No. PCT/JP2021/021810, filed on Jun. 8, 2021.

(51) Int. Cl. A61B 1/00

(52) U.S. Cl. CPC A61B 1/00057; A61B 1/00018; A61B 1/00009

(57) ABSTRACT

An image pickup unit includes: an image sensor; a physical quantity detection sensor; and a holding member that is provided with a wiring connection member to which a cable is connected, in which a distal end of the stress detection sensor is provided between the physical quantity detection sensor and the wiring connection member.

### CROSS REFERENCE TO RELATED APPLICATION

This application is a continuation application of PCT/JP2021/021810 filed on Jun. 8, 2021, the entire contents of which are incorporated herein by this reference.

### BACKGROUND OF THE INVENTION

### 1. Field of the Invention

The present invention relates to an image pickup unit provided at a distal end of an insertion portion having a bending portion, and relates to an endoscope.

### 2. Description of the Related Art

As is well known, endoscopes are widely used for observation, treatment and the like of insides of living bodies (inside body cavities), or for inspection, repair and the like of industrial plant equipment. Such an endoscope includes an insertion portion for insertion into a curved duct. Such an endoscope is known to have a configuration in which an image pickup unit or the like is provided at the distal end portion of the insertion portion.

For such an endoscope, for example, as disclosed in Japanese Patent Application Laid-Open Publication No. 11-281897, a technique is known that detects the direction of a distal end portion by providing a physical quantity detection sensor such as a gyro (angular acceleration) sensor or an acceleration sensor at the distal end portion of the endoscope.

Further, for example, Japanese Patent Application Laid-Open Publication No. 2020-137704 discloses a technique of obtaining in advance error due to variations of wiring to a position sensor included in the distal end of an endoscope, holding the information in a processor memory, and correcting the sensor output with the error information in the processor memory when the sensor is driven.

### SUMMARY OF THE INVENTION

An image pickup unit of an aspect of the present invention includes: an image sensor configured to pick up an object image; a physical quantity detection sensor configured to detect a physical quantity such as acceleration or angular acceleration; a stress detection sensor configured to detect a correction value of the physical quantity from stress in a dynamic state; a cable configured to transmit a signal from the image sensor, the physical quantity detection sensor, and the stress detection sensor; and a holding member that holds the image sensor, the physical quantity detection sensor, and the stress detection sensor, and is provided with a wiring connection member to which the cable is connected, in which a distal end of the stress detection sensor is provided between the physical quantity detection sensor and the wiring connection member.

An endoscope of another aspect of the present invention includes an insertion portion including a distal end portion and a bending portion, the distal end portion incorporating an image pickup unit, in which the image pickup unit includes: an image sensor configured to pick up an object image, a physical quantity detection sensor configured to detect a physical quantity such as acceleration or angular acceleration; a stress detection sensor configured to detect a correction value of the physical quantity from stress in a dynamic state; a cable configured to transmit a signal from the image sensor, the physical quantity detection sensor, and the stress detection sensor; and a holding member that holds the image sensor, the physical quantity detection sensor, and the stress detection sensor, and is provided with a wiring connection member to which the cable is connected, and a distal end of the stress detection sensor is provided between the physical quantity detection sensor and the wiring connection member.

### BRIEF DESCRIPTION OF THE DRAWINGS

FIG. 1 is a diagram showing an appearance of an endoscope according to an aspect of the present invention;

FIG. 2 is a diagram describing a bending state of a bending portion of an insertion portion according to the aspect of the present invention:

FIG. 3 is a top view showing a configuration of an image pickup unit to which cables are connected according to the aspect of the present invention:

FIG. 4 is a bottom view showing the configuration of the image pickup unit to which the cables are connected according to the aspect of the present invention;

FIG. 5 is a side view showing the configuration of the image pickup unit to which the cables are connected according to the aspect of the present invention:

FIG. 6 is a block diagram showing a physical quantity detection sensor, a stress detection sensor, a correction value calculation unit, and a memory according to the aspect of the present invention:

FIG. 7 is a schematic diagram showing three-axis directions of the physical quantity detection sensor according to the aspect of the present invention;

FIG. 8 is a side view showing a configuration of an image pickup unit of a first modification to which cables are connected;

FIG. 9 is a side view showing a configuration of an image pickup unit of a second modification to which cable are connected:

FIG. 10 is a side view showing a configuration of an image pickup unit of a third modification to which cables are connected;

FIG. 11 is a side view showing a configuration of an image pickup unit of a fourth modification to which cables are connected; and

FIG. 12 is a side view showing a configuration of an image pickup unit of a fifth modification to which cables are connected.

### DETAILED DESCRIPTION OF THE PREFERRED EMBODIMENT

The following describes an aspect of an image pickup unit for endoscope and an endoscope of the present invention. Note that in the following description, drawings based on an embodiment are schematic, relationships among thicknesses and widths in individual parts, ratios among thicknesses in individual parts, or the like are different from the relationships and the ratios in the actual parts, and there may be parts including different dimensional relationships and ratios among the drawings.

The endoscope in the following description on configurations can be applied to: a bronchoscope; a urological endoscope; a so-called flexible scope with a flexible insertion portion for insertion into a digestive tract or the like; and a so-called rigid endoscope, used for surgery, having a rigid insertion portion including a bending portion.

The following describes an endoscope of an aspect of the present invention based on the drawings.

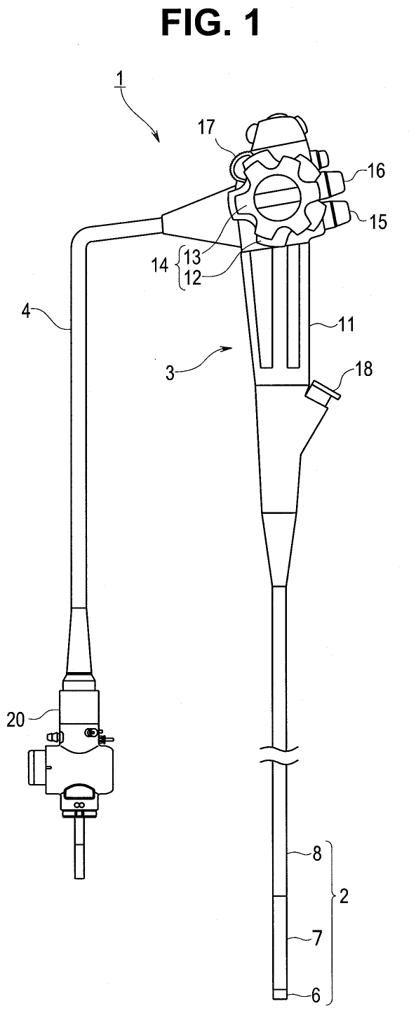

As shown in FIG. 1, an endoscope 1 according to the present embodiment includes a long and elongated insertion portion 2 to be inserted into an object, an operation portion 3, and a universal cable 4 that is a composite cable. The insertion portion 2 of the endoscope 1 includes a distal end portion 6, a bending portion 7, and a flexible tube portion 8 in order from the distal end.

A bending operation knob 14 for bending operation of the bending portion 7 of the insertion portion 2 is turnably disposed in the operation portion 3. The operation portion 3 is also provided with: switches 15 and 16 for switching various endoscope functions and observation images such as near-point observation, far-point observation, release, and still images; a fixing lever 17 for fixing turn of bending operation knob 14; or the like.

Note that the bending operation knob 14 is composed of a UD bending operation knob 12 for bending operation of the bending portion 7 in an up-down direction and an RL bending operation knob 13 for bending operation of the bending portion 7 in a left-right direction. The bending operation knob 14 is disposed so that two substantially disk-shaped rotary knobs, which are the UD bending operation knob 12 and the RL bending operation knob 13, overlap each other.

The connecting portion between the insertion portion 2 and the operation portion 3 includes: a grasping portion 11 to be grasped by a user; and a treatment instrument insertion channel insertion portion 18 placed in the grasping portion 11. The treatment instrument insertion channel insertion portion 18 is an opening portion of a treatment instrument insertion channel that allows insertion of various treatment instruments disposed in the insertion portion 2.

The universal cable 4 extending from the operation portion 3 includes a light source device (not shown) and a detachable endoscope connector 20 at the extending end. Note that the endoscope 1 of the present embodiment transmits illumination light from a light source device (not shown) to the distal end portion 6 with a light guide bundle (not shown) of illumination means inserted and disposed through the insertion portion 2, the operation portion 3, and the universal cable 4.

The endoscope connector 20 is connected to a coiled coil cable, which is not shown here. The coil cable has an extending end provided with a video processor (not shown) and a detachable electrical connector.

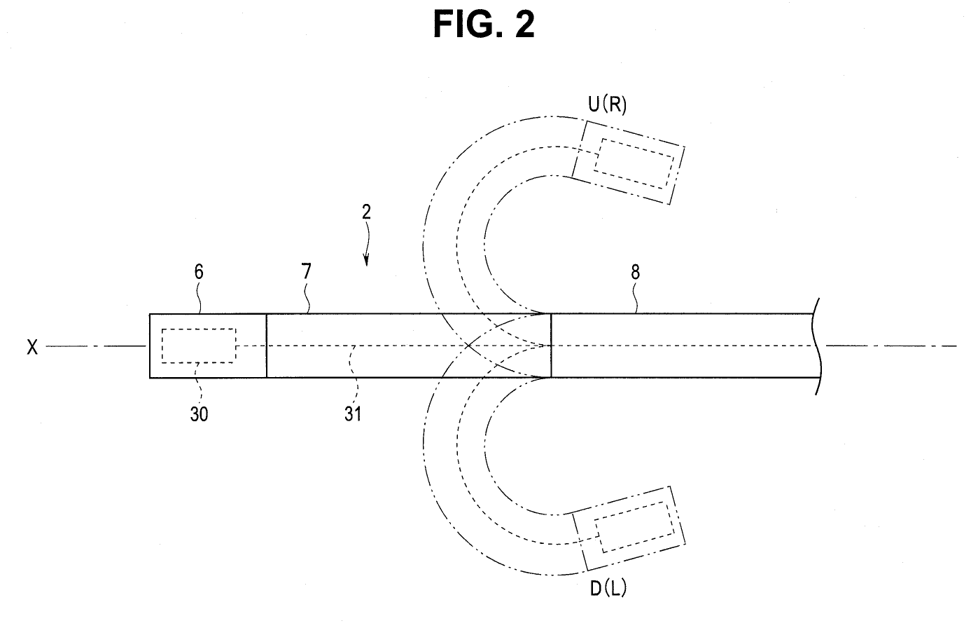

As shown in FIG. 2, the bending portion 7 of the insertion portion 2 is operated to bend in the up-down direction according to turning operation of the UD bending operation knob 12. The bending portion 7 is operated to bend in the right-left direction according to turning operation of the RL bending operation knob 13. Note that a combination of bending operations of up, down, left, and right enables the bending portion 7 to freely bend 360 degrees around the longitudinal axis X that is an insertion axis.

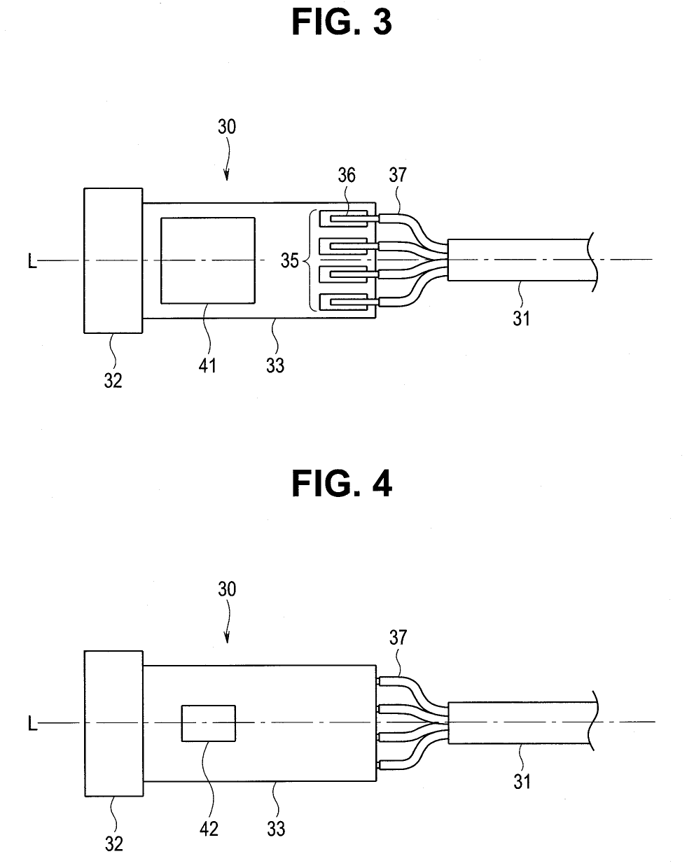

The distal end portion 6 of the insertion portion 2 incorporates an image pickup unit 30. As shown in FIGS. 3 and 4, the image pickup unit 30 includes an image pickup section 32 that is an image sensor including a CCD or CMOS configured to pick up an object image on a distal end side that is an object side, and a substrate 33 serving as a holding member placed on a proximal end side and electrically connected to the image pickup section 32. Note that the image pickup unit 30 is provided with a reinforcing frame (not shown) that covers a lens unit (not shown), the image pickup section 32, and the substrate 33. The reinforcing frame has an inside filled with curable resin that covers the surroundings of the image pickup section 32 and the substrate 33.

The substrate 33 is a mounting substrate on which rigid electronic components are mounted. The substrate 33 has one surface, which is the upper surface here, on which a physical quantity detection sensor 41 that is a MEMS sensor such as an acceleration sensor or a gyro (angular acceleration) sensor is mounted. Here, the substrate 33 has the other surface, which is the lower surface, on which a stress detection sensor 42 such as a strain sensor is mounted.

Note that the stress detection sensor 42 detects stress generated in a rigid body by detecting strain such as extension, contraction, or twisting of the rigid body such as: the substrate 33 on which the physical quantity detection sensor 41 is mounted; the curable resin that fills the space around the substrate 33; or the reinforcing frame around the resin. In other words, the stress detection sensor 42 is mounted on the substrate 33 and is integrated by fixing. As a result, the stress detection sensor 42 can estimate the stress of a rigid body including the substrate 33 on which the physical quantity detection sensor 41 is mounted, the curable resin, and the reinforcing frame.

The substrate 33 has a proximal end portion having the one surface on which four wiring connection members 35 here, which are wiring conductors, are formed by plating printing or the like. The four wiring connection members 35 are respectively connected to core wires 36 of four wires 37 of the cable 31 by soldering or the like.

Note that the cable 31 is inserted through the insertion portion 2. The cable 31 transmits signals from the image pickup section 32, the physical quantity detection sensor 41, and the stress detection sensor 42.

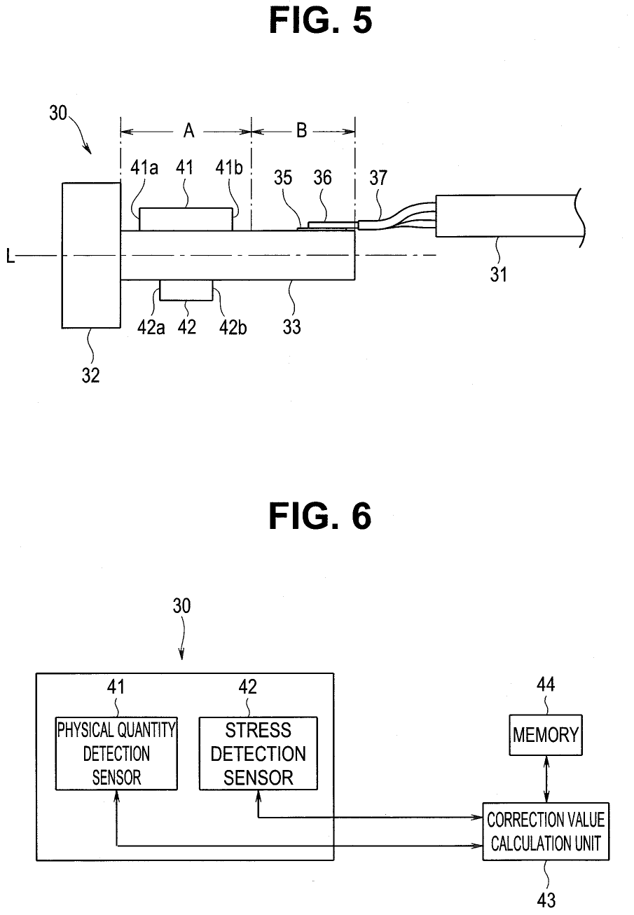

As shown in FIG. 5, the substrate 33 defines a minute deformation region A on the distal end side in a direction of the longitudinal axis L of the substrate 33 and a stress concentration deformation region B on the proximal end side in the direction of L, as deformation regions in a dynamic state according to a tension change of the cable 31 caused by bending operation of the bending portion 7.

In other words, the substrate 33 has the cable 31 connected to the proximal end portion. Therefore, when the bending portion 7 bends, the substrate 33 has a larger deformation amount with respect to a tension change of the cable 31 at the proximal end side than at the distal end side. Note that the substrate 33 defines an area in a predetermined length on the distal end side in the direction of the longitudinal axis L as a minute deformation region A, and defines an area in a predetermined length from the minute deformation region A on the proximal end side in the direction of L as a stress concentration deformation region B.

The image pickup unit 30 of the present embodiment is provided with the physical quantity detection sensor 41 and the stress detection sensor 42 each mounted on the substrate 33, on the minute deformation region A on the distal end side of the substrate 33.

The substrate 33 has the physical quantity detection sensor 41 mounted on the upper surface (front surface) closer to the distal end than the four wiring connection members 35. The substrate 33 has the stress detection sensor 42 mounted on the lower surface (rear surface) closer to the distal end than the four wiring connection members 35.

As a result, the substrate 33 has the stress detection sensor 42 placed on the surface front-rear symmetrical to the surface on which the physical quantity detection sensor 41 is placed. Therefore, the substrate 33 facilitates estimation of the load generated on the physical quantity detection sensor 41, and is allowed to shorten in the direction of the longitudinal axis L.

The physical quantity detection sensor 41 and the stress detection sensor 42 are mounted on the substrate 33 so that a distal end surface 42a of the stress detection sensor 42 is located on the proximal end side with respect to a distal end surface 41a of the physical quantity detection sensor 41.

Thus, the stress detection sensor 42 is configured to have the distal end surface 42a provided between the distal end surface 41a of the physical quantity detection sensor 41 and the four wiring connection members 35 on the substrate 33.

Such a configuration places the stress detection sensor 42 between the wiring connection member 35 near the stress generation source and the physical quantity detection sensor 41, improving the stress load detection sensitivity.

Further, the physical quantity detection sensor 41 and the stress detection sensor 42 are mounted on the substrate 33 so that a proximal end surface 42b of the stress detection sensor 42 is located on the distal end side with respect to a proximal end surface 41b of the physical quantity detection sensor 41.

The endoscope 1 has a relationship between output variations of the physical quantity detection sensor 41 and outputs of the stress detection sensor 42 according to bending states (bending angles) of the bending portion 7 of the insertion portion 2. At the time of shipping from the factory, the endoscope 1 stores correction values for the detection values of the physical quantity detection sensor 41 in the memory 44. The correction values are calculated by a correction value calculation unit 43 from the relationship (see FIG. 6).

The endoscope 1, when used, reads the correction values stored in the memory 44 according to the bending states (bending angles) of the bending portion 7, and corrects the detection values of the physical quantity detection sensor 41 with the correction value calculation unit 43. The memory 44 is provided in the operation portion 3 of the endoscope 1 or the like.

Note that the correction value calculation unit 43 may be provided on the side of the endoscope 1, or may be provided on the side of an external device such as a correction calculation device, a light source device, or a video processor.

The following describes an example of a configuration and a correction method when the physical quantity detection sensor 41 is an acceleration sensor.

Assuming that the acceleration data A obtained from the physical quantity detection sensor 41 of the acceleration sensor has a linear relationship with the actually applied acceleration a, the acceleration data A can be expressed as A=α·a+β.

Note that a is the proportional coefficient and means the sensitivity of the physical quantity detection sensor 41.

Also, β is the intercept and means the offset of the physical quantity detection sensor 41.

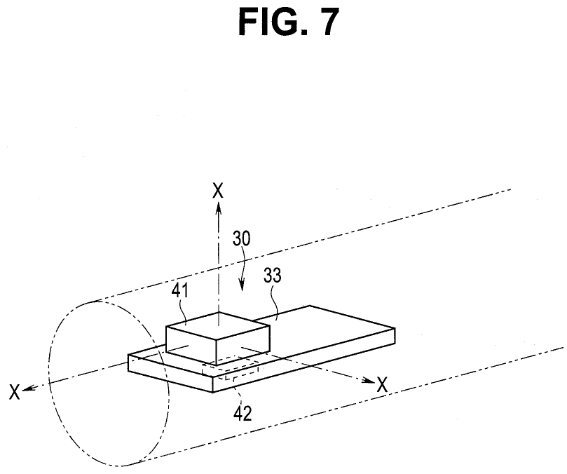

As shown in FIG. 7, the physical quantity detection sensor 41 is a three-axis sensor, so A, a, α, and β each have components (x, y, z) of each axis (the following Equation 1).

Ai(εj)=αi·αi(εj)+βi(εj)(i,j=x,y,z) [Equation 1]

Generally, the sensor output A is corrected with α and β, and if the correction is perfect, α=1 and β=0 in the relationship between A′ and the acceleration a.

By focusing on the fact that the sensitivity α and the offset β of the physical quantity detection sensor 41, which is an acceleration sensor, have dependency on the amount of strain ε, the correction is calculated from the dependency (the following Equation 2).

$${A}^{′} i ( ε j ) = \frac{Ai ( ε j ) - β i ( ε j )}{\begin{matrix}ai ( ε j ) \\ ( i , j = x , y , z )\end{matrix}} \qquad [ Equation 2 ]$$

As described above, in the endoscope 1 of the present embodiment, each output value of the physical quantity detection sensor 41 varies when a stress load is applied to the physical quantity detection sensor 41 through the cable 31 connected to the image pickup unit 30, the substrate 33 provided in the image pickup unit 30, or the like, accompanying bending operation of the bending portion 7 provided in the insertion portion 2. Then, the endoscope 1 corrects the output value of the physical quantity detection sensor 41 with the correction value stored in the memory 44 according to the strain that the stress detection sensor 42 detects according to the stress load occurring in the physical quantity detection sensor 41.

In other words, the endoscope 1 corrects the variation in each output value due to the stress load applied to the physical quantity detection sensor 41 provided in the image pickup unit 30 with a correction value corresponding to the strain detected by the stress detection sensor 42. The stress load, as the bending portion bends, is applied to the physical quantity detection sensor 41 through: the cable 31 that is disposed to extend to the distal end portion 6 of the insertion portion 2 and is connected to the image pickup unit 30; the substrate 33 provided in the image pickup unit 30; or the like.

In other words, a correction value for correcting the physical quantity detected by the physical quantity detection sensor 41 is detected from the strain output value detected by the stress detection sensor 42.

As a result, the endoscope 1 can correct the physical quantity detected by the physical quantity detection sensor 41 to improve the accuracy when a stress load is applied to the physical quantity detection sensor 41 provided on the image pickup unit 30, which detects a physical quantity for measuring the direction of the distal end portion 6, or the like, through the cable 31 connected to the image pickup unit 30, the substrate 33 on which the physical quantity detection sensor 41 is mounted, or the like.

### (Modifications)

The following describes different placement examples of the physical quantity detection sensors 41 and the stress detection sensors 42 mounted on the substrate 33 or different shape examples of the substrate 33.

### (First Modification)

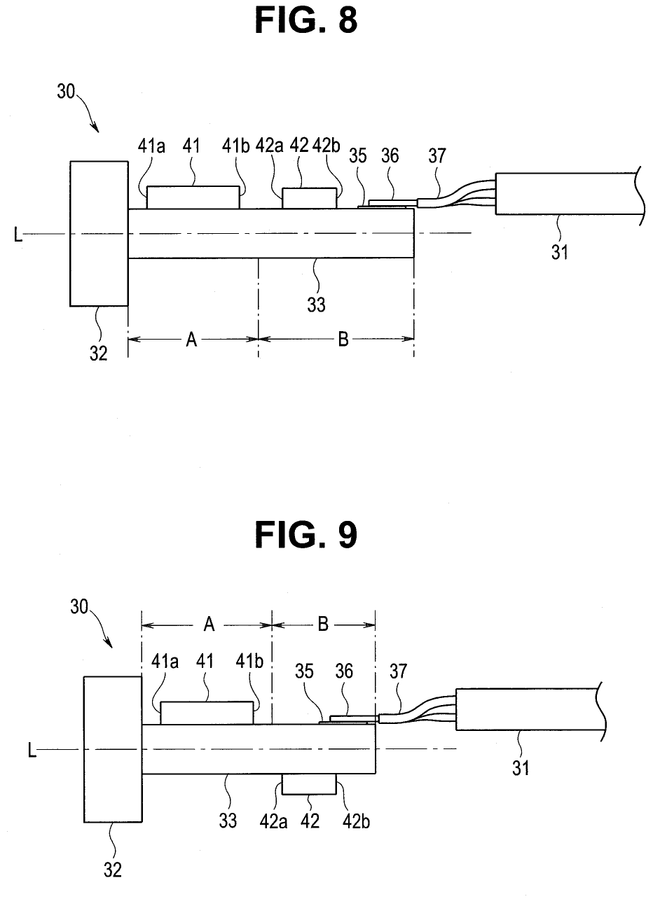

As shown in FIG. 8, the image pickup unit 30 of the present modification has the physical quantity detection sensor 41 and the stress detection sensor 42 mounted on the same surface of the substrate 33, here on the upper surface. In addition, the image pickup unit 30 has a stress detection sensor 42 placed between the physical quantity detection sensor 41 and the wiring connection member 35.

Here, the distal end surface 42a and the proximal end surface 42b of the stress detection sensor 42 are placed closer to the proximal end than the distal end surface 41a and the proximal end surface 41b of the physical quantity detection sensor 41. Further, the physical quantity detection sensor 41 is mounted in the minute deformation region A on the distal end side of the substrate 33, and the stress detection sensor 42 is mounted in the stress concentration deformation region B on the proximal end side of the substrate 33.

Such a configuration allows the stress detection sensor 42 to detect minute strain in the vicinity of the physical quantity detection sensor 41 with high accuracy.

### (Second Modification)

As shown in FIG. 9, the image pickup unit 30 of the present modification has a physical quantity detection sensor 41 mounted on the upper surface, which is one surface of the substrate 33, and a stress detection sensor 42 placed on the lower surface, which is the other surface opposite to the wiring connection member 35.

Also here, the distal end surface 42a and the proximal end surface 42b of the stress detection sensor 42 are placed closer to the proximal end than the distal end surface 41a and the proximal end surface 41b of the physical quantity detection sensor 41. Further, the physical quantity detection sensor 41 is mounted in the minute deformation region A on the distal end side of the substrate 33, and the stress detection sensor 42 is mounted in the stress concentration deformation region B on the proximal end side of the substrate 33.

Such a configuration allows the substrate 33 to prevent the length in the direction of the longitudinal axis L from increasing due to the region where the wiring connection member 35 necessary for connecting the core wires 36 of the cable 31 is provided and the region where the stress detection sensor 42 is mounted.

Note that the image pickup unit 30 of the present modification has the stress detection sensor 42 mounted on a surface of the substrate 33 different from a surface on which the physical quantity detection sensor 41 is mounted, but has no particular demerit with respect to the stress load estimation with the stress detection sensor 42.

### (Third Modification)

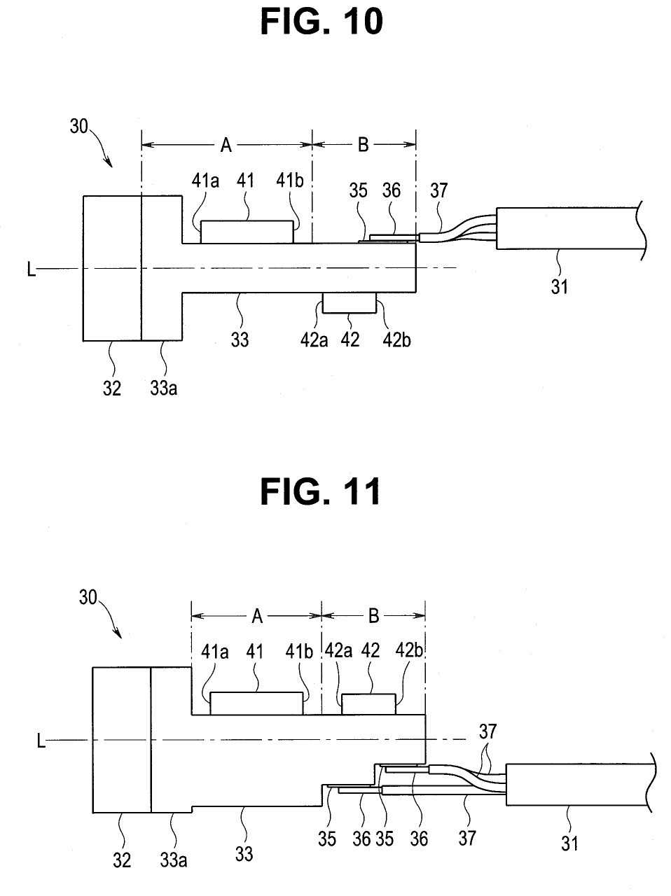

As shown in FIG. 10, the image pickup unit 30 of the present modification has a physical quantity detection sensor 41 mounted on the upper surface, which is one surface of the substrate 33, and a stress detection sensor 42 placed on the lower surface, which is the other surface opposite to the wiring connection member 35, as in the second modification.

Also here, the distal end surface 42a and the proximal end surface 42b of the stress detection sensor 42 are placed closer to the proximal end than the distal end surface 41a and the proximal end surface 41b of the physical quantity detection sensor 41. Further, the physical quantity detection sensor 41 is mounted in the minute deformation region A on the distal end side of the substrate 33, and the stress detection sensor 42 is mounted in the stress concentration deformation region B on the proximal end side of the substrate 33.

The substrate 33 is configured to be provided with a reinforcing substrate 33a bonded to the proximal end surface of the image pickup section 32 to be able to reduce the stress load in order to increase the rigidity in the vicinity of the physical quantity detection sensor 41.

Specifically, the substrate 33 has a T-shape having a reinforcing substrate 33a having a predetermined thickness in a direction orthogonal to the longitudinal axis L of the substrate 33, on the distal end side with respect to the physical quantity detection sensor 41. In addition, the reinforcing substrate 33a here has a bonding surface having a similar shape to the proximal end surface of the image pickup section 32.

Such a configuration allows the substrate 33 to improve rigidity and reduce the stress load in the vicinity of the physical quantity detection sensor 41.

### (Fourth Modification)

As shown in FIG. 11, the image pickup unit 30 of the present modification has the physical quantity detection sensor 41 and the stress detection sensor 42 each mounted on the same surface of the substrate 33, on the upper surface here, as in the first modification. In addition, the substrate 33 has a stress detection sensor 42 placed between the physical quantity detection sensor 41 and the wiring connection member 35. Further, the substrate 33 includes a reinforcing substrate 33a bonded to the proximal end surface of the image pickup section 32 in order to increase the rigidity in the vicinity of the physical quantity detection sensor 41, as in the third modification.

Also here, the distal end surface 42a and the proximal end surface 42b of the stress detection sensor 42 are placed closer to the proximal end than the distal end surface 41a and the proximal end surface 41b of the physical quantity detection sensor 41. Further, the physical quantity detection sensor 41 is mounted in the minute deformation region A on the distal end side of the substrate 33, and the stress detection sensor 42 is mounted in the stress concentration deformation region B on the proximal end side of the substrate 33.

In addition, the substrate 33 has: the minute deformation region A having a thickness set larger toward the lower surface side in the direction orthogonal to the longitudinal axis L; and the stress concentration deformation region B having the lower surface formed in a stepped shape, on which a plurality of the wiring connection members 35 are formed.

Such a configuration allows the substrate 33 to have further increased rigidity, and have the wiring connection members 35 formed on the stepped surface so that the wiring connection members 35 are provided on a plurality of rows in the direction of the longitudinal axis L when the number of wirings 37 is large.

### (Fifth Modification)

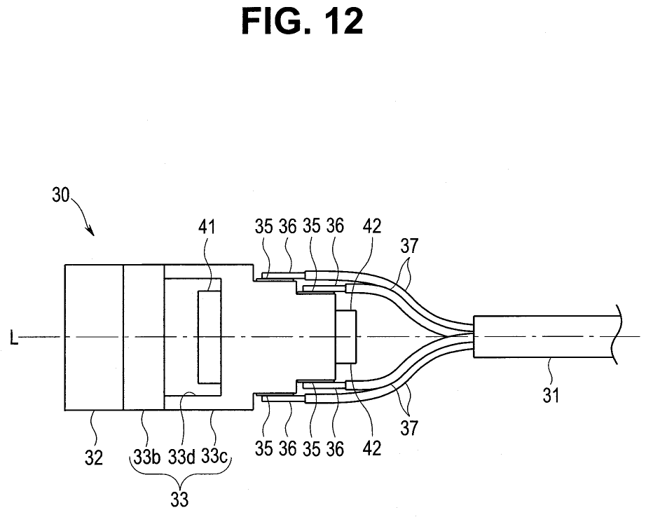

As shown in FIG. 12, the image pickup unit 30 of the present modification includes a main substrate 33c having a recess 33d that opens on the distal end side of the substrate 33, and a sub substrate 33b that closes the recess 33d and is bonded to the image pickup section 32, to further increase rigidity.

Also, the stress detection sensor 42 is provided on the proximal end surface of the main substrate 33c. The proximal end portion of the main substrate 33c is formed in a stepped shape on either both the upper and lower surfaces or both the left and right surfaces, and a plurality of wiring connection members 35 are formed on the stepped surface.

Such a configuration enables the substrate 33 to surround the physical quantity detection sensor 41 with the substrate materials of the main substrate 33c and the sub substrate 33b to receive a higher load from the cable 31.

The invention described in the above embodiment and the modifications is not limited to the embodiment and the modifications, and various modifications can be made in the implementation stage without departing from the gist of the invention. Furthermore, the above-described embodiment and modifications include inventions at various stages, and various inventions can be extracted by appropriate combinations of a plurality of the disclosed components.

For example, if some components are deleted from all the components shown in the embodiment and the modifications and if the stated problem can be solved and the stated effect can be obtained, the configuration in which these components are deleted can be extracted as the invention.

What is claimed is:

1. An image pickup unit comprising:
an image sensor configured to pick up an object image;
a physical quantity detection sensor configured to detect a physical quantity such as acceleration or angular acceleration;
a stress detection sensor configured to detect a correction value of the physical quantity from stress in a dynamic state;
a cable configured to transmit a signal from the image sensor, the physical quantity detection sensor, and the stress detection sensor; and
a holding member that holds the image sensor, the physical quantity detection sensor, and the stress detection sensor, and is provided with α wiring connection member to which the cable is connected,
wherein a distal end of the stress detection sensor is provided between the physical quantity detection sensor and the wiring connection member.

2. The image pickup unit according to claim 1, wherein the distal end of the stress detection sensor is located closer to a distal end than a proximal end of the physical quantity detection sensor.

3. The image pickup unit according to claim 1, wherein a proximal end of the stress detection sensor is located closer to a proximal end than a proximal end of the physical quantity detection sensor.

4. The image pickup unit according to claim 1, wherein the stress detection sensor is provided on a same surface of the holding member as one surface on which the physical quantity detection sensor is provided.

5. The image pickup unit according to claim 1, wherein the stress detection sensor is provided on another surface of the holding member, the other surface being opposite to one surface on which the physical quantity detection sensor is provided.

6. The image pickup unit according to claim 1, wherein the wiring connection member is located closer to a proximal end than the distal end of the stress detection sensor.

7. An endoscope comprising an insertion portion including a distal end portion and a bending portion, the distal end portion incorporating an image pickup unit,
wherein the image pickup unit includes:
an image sensor configured to pick up an object image;
a physical quantity detection sensor configured to detect a physical quantity such as acceleration or angular acceleration;
a stress detection sensor configured to detect a correction value of the physical quantity from stress in a dynamic state;
a cable configured to transmit a signal from the image sensor, the physical quantity detection sensor, and the stress detection sensor; and
a holding member that holds the image sensor, the physical quantity detection sensor, and the stress detection sensor, and is provided with α wiring connection member to which the cable is connected, and
wherein a distal end of the stress detection sensor is provided between the physical quantity detection sensor and the wiring connection member.
